(19) United States
(12) Patent Application Publication
(10) Pub. No.: US 2006/0211764 A1
(43) Pub. Date: Sep. 21, 2006

(54) Cell activator, process for producing cell activator and apparatus therefor

(76) Inventors: Yoichiro Nagasu, Atugi-shi (JP); Shin Aizawa, Tokyo (JP); Toshihisa Hoshiai, Tokyo (JP); Tadatomo Nagao, Kumamoto (JP); Shouichi Nonaka, Kumamoto (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/533,360

(22) PCT Filed: Oct. 31, 2003
(86) PCT No.: PCT/JP03/14024

§ 371 Date: Apr. 10, 2006

(30) Foreign Application Priority Data
Nov. 1, 2002 (JP) 2002-3205070

(51) Int. Cl. A61K 31/365; F28D 7/00; C07D 323/02

(52) U.S. Cl. 514/450; 549/267; 422/200

(57) ABSTRACT

It is determined whether phase noise and parasitic oscillation take place on the basis of a carrier-to-noise (C/N) ratio calculated with amplitudes in an amplitude direction of signal points of a reception signal and a C/N ratio calculated with amplitudes in a phase direction of the signal points of the reception signal. Further, when it has been determined that phase noise and parasitic oscillation take place, an alarm is displayed on a screen. When it has been determined that phase noise and parasitic oscillation take place, the phase of a demodulation circuit is compensated based on the detected phase noise.

### TECHNICAL FIELD

This invention relates to the cell activator to activate cells, manufacturing methods for the cell activator, and the instruments for them.

### BACKGROUND ART

The inventor discovered this phenomenon in 1982—that is, the supernatant fluid of a culture solution, which had been used to culture cancer cells, inhibits the growth of cancer cells. Then, by analyzing the substance in the culture solution, his study concluded that it was cyclic polymerize lactic acid. From the viewpoint that cyclic polymerize lactic acid is effective against as a drug tumors; his studies in cooperation with other scientific researchers have produced countless good results.

The results of such studies led to numerous patent applications, for instance, Japanese published unexamined application (hereinafter referred to as Toku-kai-hei official report) No.05-310581, Toku-kai-hei No.06-336427 official report, Toku-kai-hei No.07-233061 official report, Toku-kai-hei No.09-227388 official report, Toku-kai-hei No. 10-130153 official report and Toku-kai No.2000-072680 official report. Described in the above-mentioned Toku-kai-hei No.05-310581 official report and Toku-kai-hei No.06-336427 official report is a cell proliferation inhibitor, which consists of a mixture of L-lactic acid straight chain condensate; condensation rate 5-23, and L-lactic acid cyclic condensate; condensation rate 2-15, against malignant tumors in animals (including human), especially against human uterine cervical cancer, human oral cavity fundus cancer, murine pulmonary carcinoma, yoshida sarcoma, rabbit hepatocellular carcinoma, human gastric cancer, thyroidal carcinoma, pulmonary carcinoma and uterine cancer.

Described in Toku-Kai-Hei 07-233061 official report is the manufacturing method for the oral ingestion agent possessing the inhibition action on the proliferation of malign tumor cells in animals including humans. This manufacturing method includes a process whereby a reaction solution containing a mixture of L-lactic acid straight chain condensate; condensation rate 3-25, and L-lactic acid cyclic condensate; condensation rate 2-15.

Described in the above-mentioned Toku-kai-hei No.09-227388 official report is an anti-malignant tumor agent, which has cyclic and straight chain mixed L-lactic acid oligomer; condensation rate 9-19 as the principal component, and which is used for either colon cancer, esophageal cancer or breast cancer.

Described in the above-mentioned Toku-kai-hei No. 10-130153 official report is an anti-malignant tumor agent, which has cyclic and straight chain mixed poly L-lactic acid; condensation rate 3-19 as the principal component, and which is used for either colon cancer, esophageal cancer or breast cancer.

Described in the above-mentioned Toku-kai No.2000-072680 official report is an immunomodulator agent, which is a cyclic and linear oligomer substance. Their chemical compositions are (C₃H₄O₂) and {(C₃H₂O)_{z}—H₂O} (where, z=2-23), respectively. Their molecular structures are of 2 types; one of the types is a zigzag cyclic structure, and the other is clathrate, which is, almost a zigzag-letter C-shaped chain structure.

Furthermore, although the inventor was not involved, described in Toku-kai No.2000-239171 official report is an oral QOL improvement agent for cancer patients, which contains cyclic and straight chain mixed poly L-lactic acid; condensation rate 3-19.

### DISCLOSURE OF INVENTION

All of the inventions described in the above official reports devises were created from the viewpoint that straight chain and cyclic mixed poly L-lactic acids, which are the condensate in what is claimed, are effective for specific diseases.

After several years of studies, the inventor recognized the necessity of returning to the starting point to verify whether or not this viewpoint was correct.

This invention resulted from a study carried out based on the above-mentioned recognition, an objective of which is to provide a cell activation agent for unspecific cells in unspecific solids, i.e., a cell activation agent, which is not species specific.

At the same time, an objective of the invention is to provide a manufacturing method of cell activation agents and the instruments.

To achieve the objectives above, the cell activator of this invention consists of cyclic polylactic acid obtained by raising the temperature of the L-lactic acid while jetting inert gas directly into it for dehydration and polymerization.

In addition, the manufacturing method for this cell activator comprises 1) the process in which a L-lactic acid solution, in which a catalyst does not exist, are introduced, 2) the process in which the cyclic polylactic acid is obtained by raising the temperature of the L-lactic acid step-wisely up to the temperature exceeding the boiling point of L-lactic acids while jetting inert gas directly into the said L-lactic acid solution for dehydration and polymerization, and 3) the process in which the obtained cyclic polylactic acid is removed from the said container.

Furthermore, the manufacturing method for this cell activator is comprised of the stirring of the said L-lactic acid solution by jetting inert gas directly into it. Moreover, the manufacturing method for this cell activator is comprised of step-wisely vacuuming when the temperature of the said L-lactic acid is raised step-wisely to the temperature exceeding the boiling point of the said L-lactic acid for dehydration and polymerization.

The instrument for manufacturing this cell activator comprises 1) main body of the vessel containing,an inlet for L-lactic acids,inert gas directly jet tube connection part and cyclic polylactic acid extract window,2) inert gas directly jet tube, which jets inert gas directly into the L-lactic acids inside the said main body of the vessel, and 3) heater to heat L-lactic acids in the said main body of the vessel.

Furthermore, this instrument for manufacturing the cell activator is comprised of possessing a built-in condenser equipped with a heat exchanger, one end of which is connected to the said main body of the container and the other end of which to the vacuum pump, and the said heat exchanger possessing a water vapor tank on which an extract window for water vapor is installed at the lower part.

### BRIEF DESCRIPTION OF DRAWINGS

FIG. 1 shows a front view of the instrument for manufacturing L-lactic acid oligomer.

FIG. 2 shows a plane view of the instrument above.

FIG. 3 shows a left sided view of the instrument above.

FIG. 4 shows a left sided view of the instrument above.

FIG. 5 shows a molecular structure model of a cyclic polymerize lactic acid at a low rate of polymerization.

FIG. 6 shows a molecular structure model of cyclic polymerize lactic acid, at a high rate of polymerization.

FIG. 7 shows a result of ultrasonography of uterine myoma. Initial state (there is an ovarian cystic hygroma, which is approximately 5 cm. It was suspected to be a malignant tumor, as a prominent part was found in part of the cyst) (Photograph 1)

FIG. 8 is after taking 30-40 g/day of CPL (reduced to approximately 4 cm) (Photograph 2)

FIG. 9 shows the result of a preoperative MRI examination. According to this, the cyst has disappeared and thus, the judgment was made that no operation was required (Photograph 3).

FIG. 10 shows a result of ultrasonography of ovarian myoma. Initial state (the ovary is swollen to approximately 6 cm). (Photograph 4)

FIG. 11 is after 3 weeks. (The ovary is further swollen to 6.5 cm). (Photograph 5)

FIG. 12 is 2 weeks after CPL administration (the ovarian cyst is just below 4 cm). (Photograph 6)

FIG. 13 is 2 weeks after further administration (the cyst had disappeared completely). (Photograph 7)

FIG. 14 shows a result of magnetic resonance analysis for health foods and pharmaceutical agents.

FIG. 15 shows variations in the results of magnetic resonance analysis of a patient suffering from pulmonary carcinoma, after taking CPL.

FIG. 16 shows a criterion for the biogenic activity determined based on by measured values by magnetic resonance analysis.

### BEST MODE FOR CARRYING OUT THE INVENTION

In order to provide for a practical use, dissolve or suspend a liquefied cyclic polymerization lactic acid in an appropriate solvent aseptically so that it reaches the specified concentration.

In the event it is used as an oral agent, the dried stock powder can be used as it is however; calcium lactate, calcium carbonate, mannitol, sorbitol, etc. are added in consideration of the characteristics of the substance. Furthermore, it can be ingested in a state of being formed as a mixture with other pharmacokinetic substances. The possible formulations have a variety from powder, granule, tablet, sugar-coated tablet, capsule, suspending agent, emulsion, etc. It can also be used as an ointment by dissolving cyclic polylactic acids in a flux such as propylene glycol, etc. before they solidify. As concrete examples of this invention, a few examples of implementation are described however; this invention is not limited to these.

<Instrument for Manufacturing>

The shoulder part of the main body of the sphere-shaped container 3 of 50L encased in the mantle heater 2 installed on the supporting frame 1 is divided equally into 4 from a plane view. Installed on these 4 parts is an inlet for L-lactic acids 4, insertion window for the thermometer 5, insertion window for the nitrogen gas direct spurting nozzle 6, and ventilation window 7, in this order. The extract window for polymerized lactic acids 13 is installed on the lower part of the main body of the sphere-shaped container 3. The heat amount of the said mantle heater 2 is decided to be 4.2 kW/h.

Adjacent to the side of the main body of the sphere-shaped container 3 is the distillation tower 8. The upper part of this distillation tower 8 is connected to the vacuum pump via the cooling trap device. The lower part is the retention part for distilled water 9, which retains and emits distilled water. The said cooling trap device is not illustrated however; it is desirable to be installed to prolong the life span of the vacuum pump, because it is used to efficiently collect water vapour and toxic vapour emitted from the distillation tower inside the vacuum system and to radically reduce the amount of vapour inhaled by the vacuum pump. For the vacuum pump, those capable of vacuuming the inside of the main body of the container 3 at least to 0.5 kPa, are used. 10 is a drain valve.

The ventilation window 7 of the main body of the container 3 and the shoulder part of the retention part for distilled water 9 installed at the lower part of the distillation tower 8 are connected with the connecting tube 11. Inside the distillation tower 8, the cooling coil 12 to which both ends of these are connected, is encased. For this cooling water circulation device, those with the temperature adjustment range of −10-25° C., cooling capacity of 2440-2900 w, and maximum flow rate of 24.5-28L/minute, are used. Inside the main body of the container 3, the temperature 14 and the nitrogen gas spurt nozzle 15 are inserted into the said insertion window each, 5 and 6 and fixed. The edge is installed at a position from which the inside wall of the main body of the container 3 can be viewed.

The basilar part of the nitrogen gas spurt nozzle 15 is connected to the liquefied nitrogen cylinder (not illustrated) via a flexible tube. Vaporized nitrogen gas from the liquefied nitrogen cylinder is directly sprayed from the edge of the nitrogen gas spurt nozzle 15 into the reaction solution inside the main body of the container 3 at a flow rate of 1-2L/minute, and thus the spurt flow per se of nitrogen gas stirs the reaction solution. Therefore, sufficient inert gas can be provided into the reaction solution. To sum up, the flow rate of this inert gas is adequate if the reaction solution bumps inside the main body of the container 3. As shown above, this invention is structured in a way that nitrogen gas is directly spurted into the reaction solution from the nitrogen gas spurt nozzle and therefore, the conventional laboratory-level condensation reaction can be progressed in bulk to realize an industry-level production.

Moreover, a stirrer is not required in this invention.

Water vapour contained in the air inhaled by the vacuum pump and transpired from the L-lactic acid solution inside the main body of the container, which pass through the distillation tower 8, is cooled by the cooling coil 12, condensed, and retained in the retention part for distilled water 9. The distilled water retained in the retention part for distilled water 9 is emitted from the drain valve 10.

<Example of Manufacturing>

Pour a certain amount, for instance, 30-40L, of L-lactic acids into the upper area of the main body of 3 sphere-shaped containers encased in the mantle heater 2.

Next, directly spray nitrogen gas into the said L-lactic acid from the nitrogen gas spurt nozzle 15 at 2L/minute, electrify the above-mentioned mantle heater 2 while stirring L-lactic acids using the spray flow from the nitrogen gas spurt nozzle 15, remove released water by vacuuming and heating for 5-7 hours to approximately 180° C., and finally heat to 200° C. to obtain cyclic polylactic acids, which are the product of the reaction. The obtained cyclic polylactic acids solidify in a sticky form at 50-60° C. and therefore, remove it when it has cooled down to approximately 100° C. from the extract window 13 of the main body of the sphere-shaped container 3.

When it is used as a preparation for injections, it was dissolved or suspended in an appropriate solvent aseptically so that it reaches a specified concentration.

When it is used as an oral drug, calcium lactate, calcium carbonate, mannitol, sorbitol, etc. are added to this stock solution before it solidifies, to solidify in a board-form. The solidified substance is crushed into powder of 30 mesh and under, dried, and stored. It can be ingested in a state of being formed as a mixture with other pharmacokinetic substances.

The possible formulations have a variety from powder, tablet, sugar-coated tablet, capsule, suspending agent, emulsion, etc. It can also be used as an ointment by dissolving in a flux such as propylene glycol, etc. before the cyclic polylactic acids solidify.

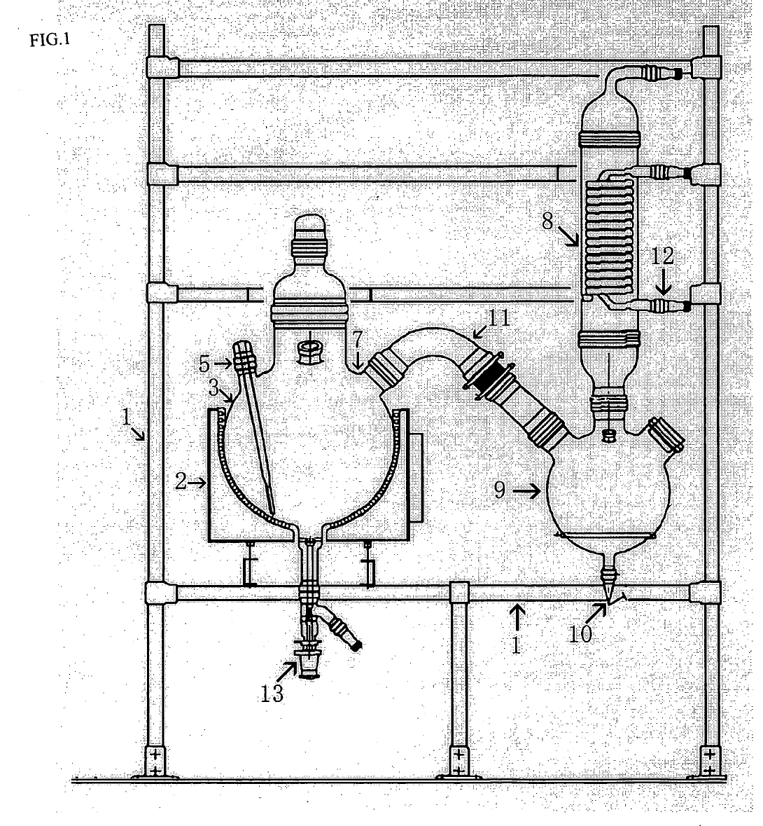

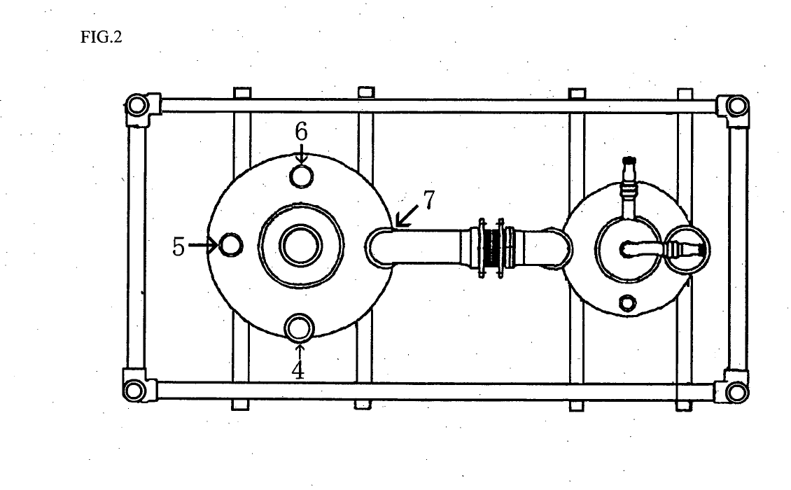

The chemical structural formula for cyclic polymerization is shown in FIG. 1 below:

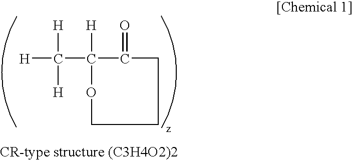

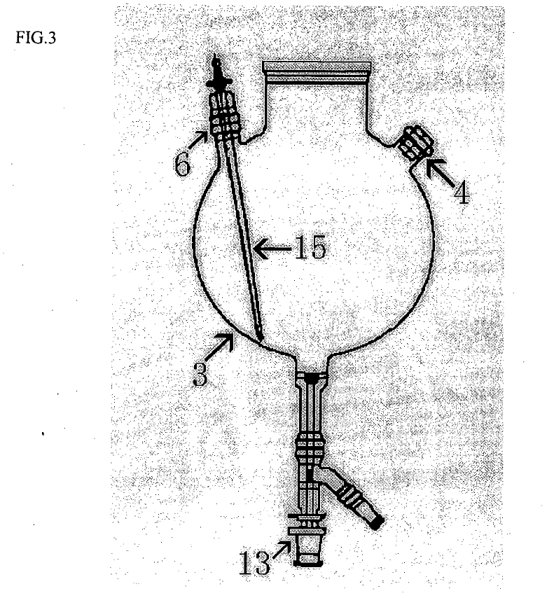

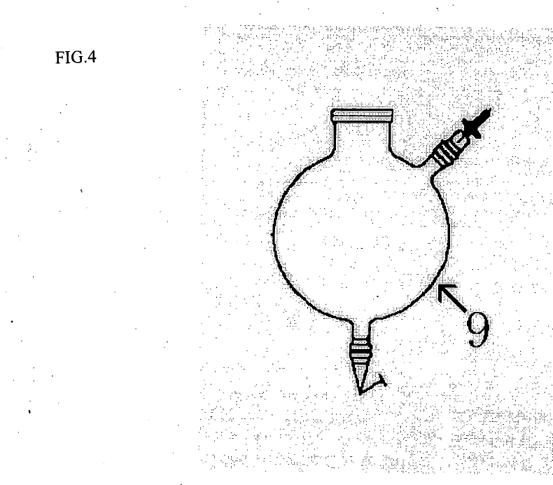

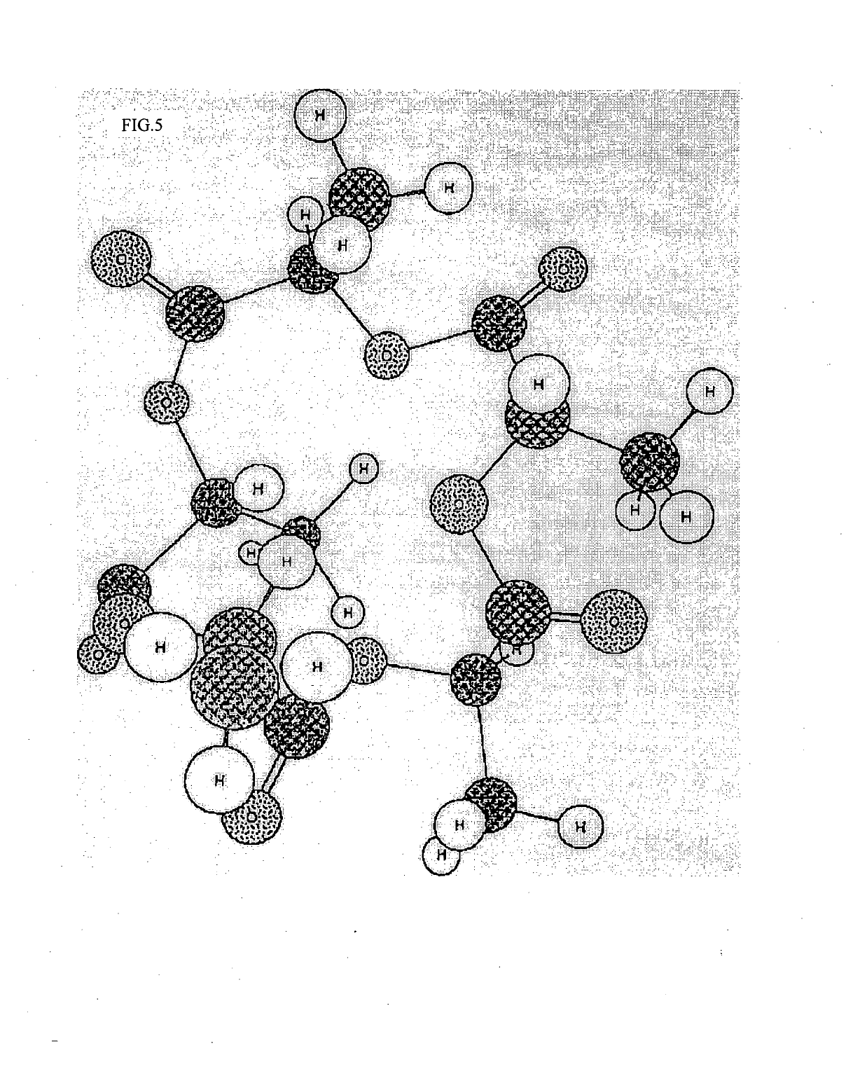

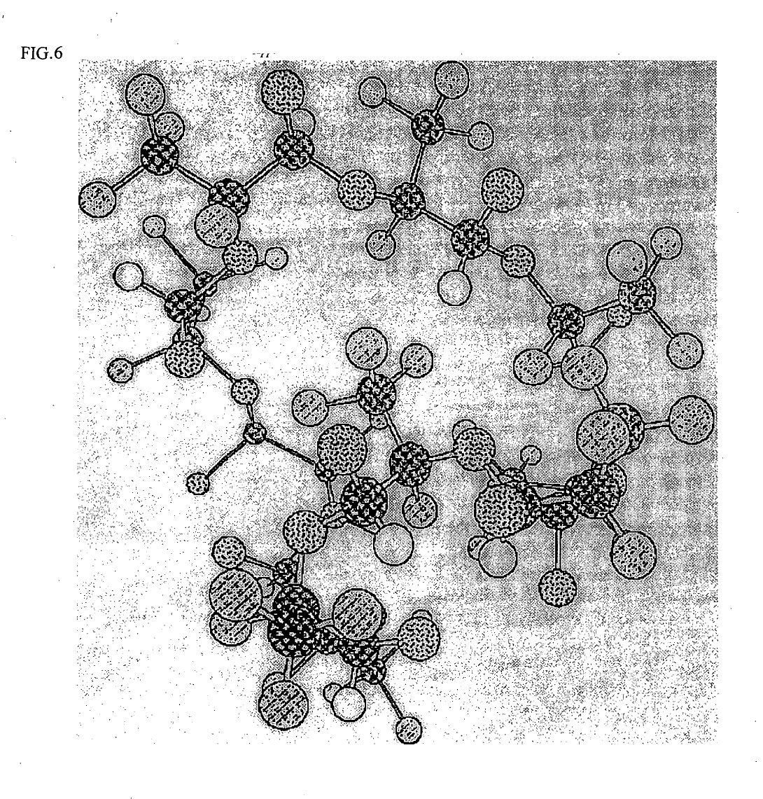

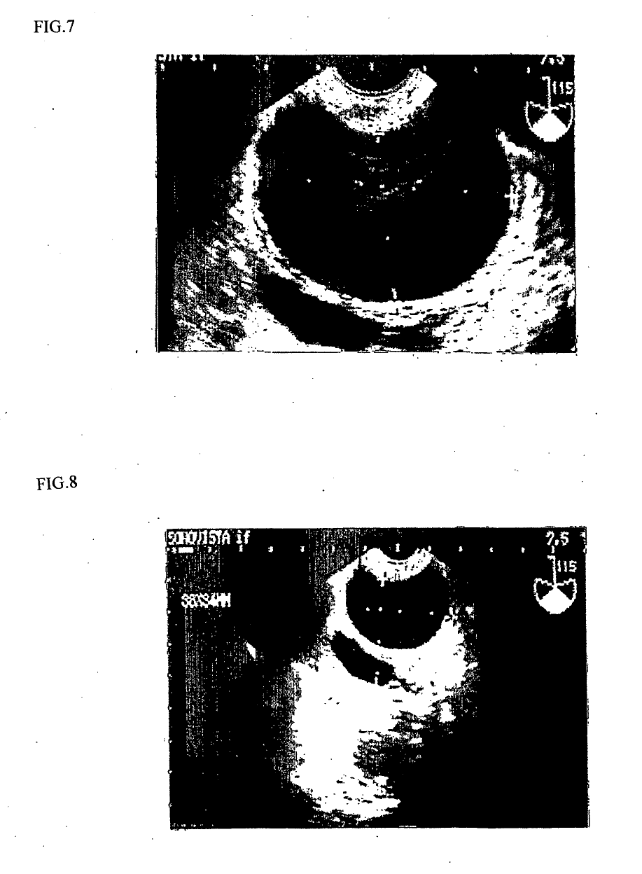

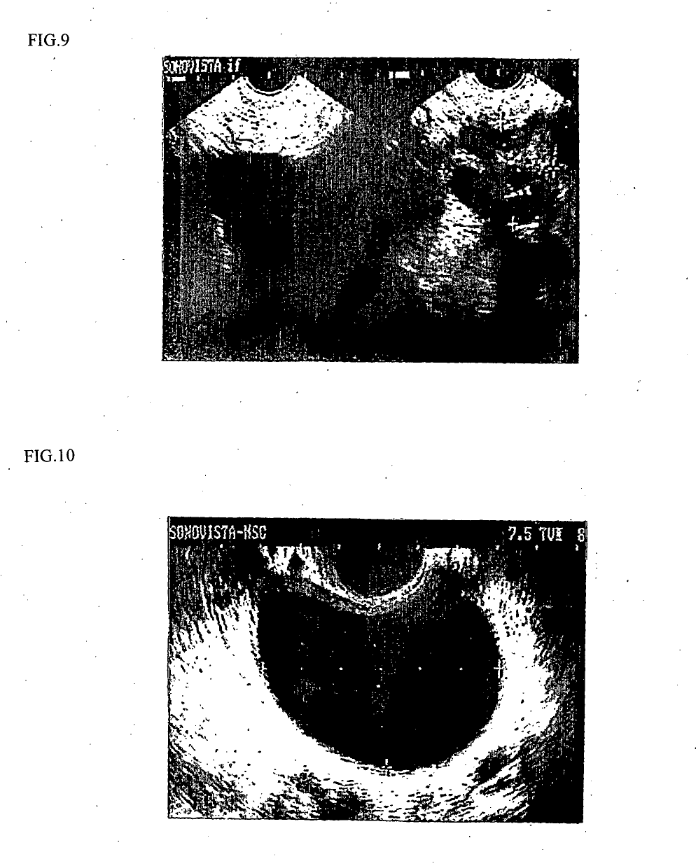

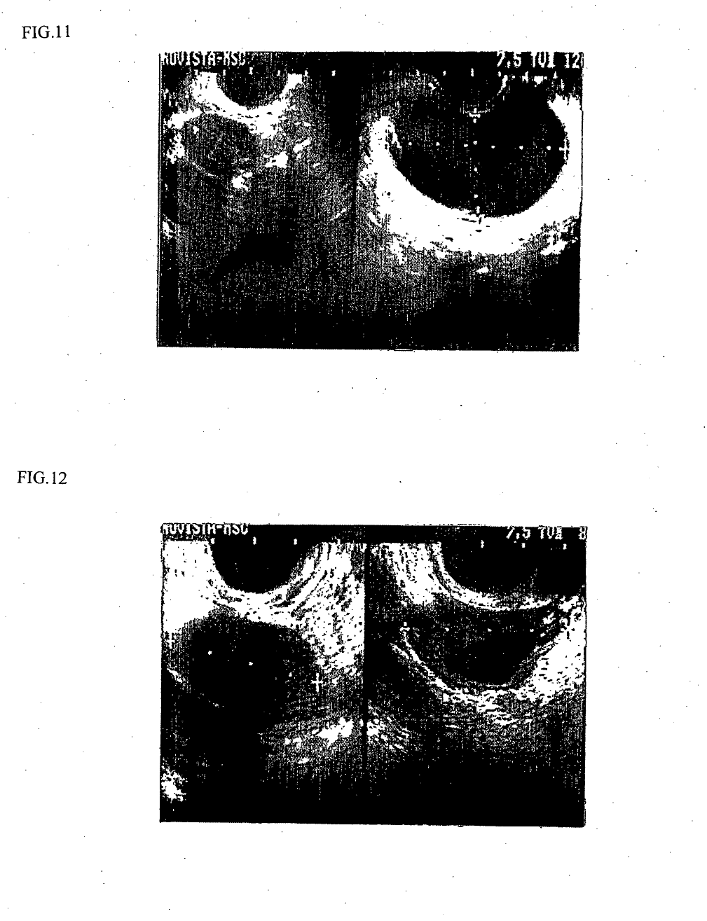

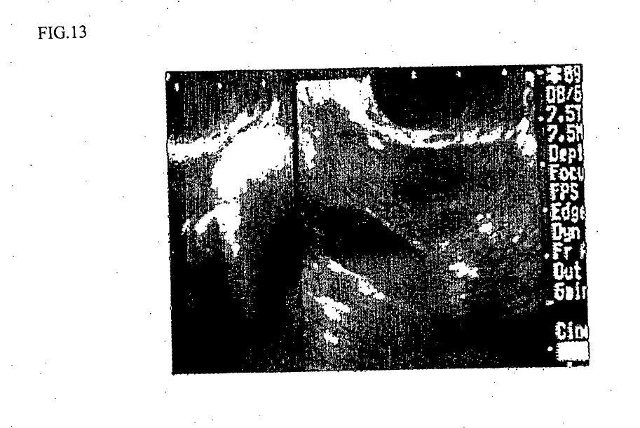

From the above, the inventor named this substance “Cyclic Poly-lactate (CPL)”. Based on the calculation of the molecular energy using a computer model, the CPL molecule indicates a zigzag closed cyclic ring structure of a low rate of polymerization (FIG. 1) while it indicates a zigzag structure in which a closed oval-shaped ring is bent like a “C” letter at a high rate of polymerization (FIG. 3). This indicates that it possess characteristics of both ring and chain.

<Example of Administration to Humans>

(Pancreatic Cancer)

A patient suffering from pancreatic cancer considered incurable because cancer cells have wound round the artery was administered 30 g/day of CPL. In approximately 1 and a half months, the patient recovered.

(Gastric Cancer)

Male patient, gastric cancer had metastasized in the liver; countless cancer cells metastasized and dispersed in the liver. The patient was administered CPL for 2 weeks, all of the countless cancer blocks in the liver, which varied in size, necrotized.

(Gastric Cancer)

Gastric cancer was detected as a result of a detailed examination, extirpated soon after the discovery, cancer as large as a head of a fetus was removed. Scattered additionally in the peritoneal cavity: Life expectancy was 3-6 months. The patient was administered 10 g/day of CPL for 5 months up to now, no sign of a recurrence.

(Gastric Cancer)

75 year old male, metastasized in the pancreas and peritoneum. Terminal stage, inoperable, 30 g/day of CPL have been administered for 150 days, progressing satisfactorily.

(Ovarian Cyst)

A case of a 40 year old patient whose uterus was extirpated due to uterine myoma. This patient has had two celitomies with a caesarean section. She received an examination for cancer because abnormal bleeding has continued recently. An ultrasonography carried out as part of this examination revealed an ovarian cyst of approximately 5 cm. Moreover, a prominent part was found in part of the cyst and it was suspected to be malign (Photograph (1)).

Therefore, she was administered 30-40 g/day of CPL in the period from the examination to the operation. The size of the ovarian cyst measured with ultrasonography carried out a few days later was reduced down to approximately 4 cm (Photograph (2)). A preoperative MRI examination revealed the cyst to have disappeared and thus, the operation was no longer required (Photograph 3).

(Ovarian Cyst)

A case of a 32 year old female patient whose wedding day was drawing close. Although it appeared not to be malign, ultrasonography revealed that an ovary had swollen up to approximately 6 cm (Photograph 4) and the patient had subjective pain. In consideration that hormone abnormalities were the cause, follow-ups were made for 2-3 weeks. Carrying out a subsequent examination, the ovary was further swollen to 6.5 cm (Photograph 5). Therefore, the patient was administered CPL for 2 weeks, with a future operation also in mind. Following ultrasonography showed that the cyst, which was as big as 6.5 cm before had shrunk to just below 4 cm (Photograph 6). Another examination was carried out 2 weeks after further administration, which showed that the cyst had disappeared completely (Photograph 7).

(Uterine/Ovarian Cancer)

44 year-old female, two years ago, the uterus was extirpated, the ovary and fallopian duct were resected and chemotherapy was performed 3 times. During hospitalization, an ileus tube was inserted due to an ileus. Then intestinal liquid and gas were emitted. A central nutrition tube was inserted into the right subclavian vein because oral ingestion was impossible. An artificial anus and urinary duct tube were attached due to a tumor in the left abdomen. Defecated from the artificial anus after 2 g/dose of CPL was ingested. Ingestion of 4-6 g/day of CPL was continued from the next day. It had a significant effect as excretion of gas, defecation, and the amount of urine increased. The ileus tube was removed 3 days later and 7 days later, oral ingestion (15-20 g/day of CPL) and eating were commenced as ileus symptoms were negative. Cancerous pain ameliorated and the ingestion of solid food from 3/10- 5/10- 7/10-whole porridge was enabled. Discharged from the hospital 3 months later. No recurrent ileus symptoms, satisfactory progress.

(Liver Cancer)

Male, administered 20 g/day of CPL for 150 days, cured (Liver cancer originated from liver cirrhosis)

Male, administered 20 g/day of CPL for 210 days, cured

(Breast Cancer)

After extirpation, metastasized to the eye. Administered 20 g/day of CPL for 210 days, satisfactory progress

(Breast Cancer)

50 year old, metastasized to the bone marrow. Administered 20 g/day of CPL for 210 days, the tumor marker decreased from 200 to 5.

(Bladder Cancer)

Male, resection was performed 5 times, administered 20-30 g/day of CPL for 50 days, no abnormal findings from examinations, nor a recurrence.

(Bladder Cancer)

57 year old male, administered 40-50 g/day of CPL for 270 days, currently at the stage of follow-ups, improved from the terminal stage.

(Laryngeal Cancer)

65 year old male, administered 20 g/day of CPL for 150 days, cured

(Tongue Cancer)

25 year old female, administered 20 g/day of CPL for 120 days, the cancer completely disappeared.

(Pulmonary Cancer Metastasis Lymphoma)

71 year old male, administered 15 g/day of CPL for 180 days, satisfactory progress

(Subclavian Lymphoma)

Female, Administered 50 g/day of CPL for 90 days, CA 15 decreased from 70 to 20.3 and CEA from 20.4 to 5.4.

(Cervical Malign Lymphoma)

39 year old male, Administered 20 g/day of CPL for 360 days, cured (colon cancer) 60 year old female, several tumors of 2 cm in size (Stage 2-3) were detected. Administered 20 g/day of CPL for 150 days, the tumors disappeared in 2 weeks. No abnormalities were found as a result of an examination carried out 5 months later.

(Leukemia)

50 year old female, administered 15-20 g/day of CPL for 170 days, cured. No recurrence detected for 2 years onwards.

(Leukemia)

66 year old male, administered 15 g/day of CPL for 5 months, cured (leukemia) 63 year old male, administered 15 g/day of CPL for 220 days, cured.

(Adverse Reactions and Control Of pain)

In the case of elderly people whose metabolic functions are weaker, ingesting a large amount of a drug often causes adverse reactions and invites risks. However, it was confirmed that CPL does not cause any adverse reactions to a 78 year old patient suffering from uterine cervical cancer who took 10 g/day. Furthermore, patients suffering from advanced cancer usually complain about pain however, ingesting CPL diminishes the pain rapidly. This patient was taking a strong painkiller when fighting against cancer, which could be replaced by a common painkiller after taking CPL. This suggests that CPL has a significant effect on ameliorating pain.

(Endometriosis)

40 year old female, with severe menstrual pain (menorrhagia), took 10 g/day of CPL orally, the next menstrual pain ameliorated, symptoms of menorrhagia also ameliorated. Such an effect was observed in 10 or so other patients. Even if the effect is less significant, at least the amount of painkiller taken has decreased. Moreover, CA125 of tumor marker also improves in the majority of the patients.

(Infertility)

37 year old female, received an examination due to menstrual pain and endometriosis, ovarian cyst detected, 6 g/day of CPL was taken because of lower abdominal pain other than menstrual pain, the pain was ameliorated within the next 2-3 days and systemic conditions had become satisfactory. After the symptoms of endometriosis ameliorated, the patient naturally conceived. After this case, CPL was administered against infertility to 10 patients, significant effects were observed with 6-9 g/day and 8 of them have conceived.

(Diabetes/Rheumatism/Hypertension)

80 year old female, took 15 g/day of CPL for 30 days, after increasing the dose up to 20 g/day and making follow-ups, dialysis was not necessary.

(Hepatitis C)

60 year old male, took 20 g/day of CPL for 16 months, satisfactory condition, shadow on the liver disappeared.

(Infantile Asthma, Atopic Dermatitis)

1 year old boy was administered with a CPL solution, which was prepared by dissolving a usual powdered preparation in drinking water so that a small child could take it. In addition to the said solution, a spraying agent was used. The said spraying agent was prepared by filling a spraying container with a solution, which was prepared by further diluting by a flux the ointment manufactured by dissolving cyclic polylactic acids in a flux such as propylene glycol, etc. before they solidify, which was described in the <Example of manufacturing> section. The dose of CPL was 15 g/day for the initial 40 days and 10 g/day for the next 140 days, in terms of the typical powdered CPL. The asthmatic seizures stopped and there was no sign of them afterwards, as a result of taking the above. In addition, eczema on the back and face disappeared as well as itchiness was cured.

(Bone-Thinning Disease Osteoporosis)

71 year old female took 6 g/day of CPL for 180 days, the bone density has increased.

(Alveolar Pyorrhoea)

Having used 200 g of CPL toothpaste prepared by mixing the ointment (CPL 1: flux 1) and toothpaste at a rate of 1:1, twice a day, alveolar pyorrhoea accompanied by bleeding was cured.

CPL is effective in controlling the proliferation of periodontal disease bacteria.

(Dermal Disease)

Having used approximately 50 g of an ointment with low viscosity (CPL 1: Flux 3.5) by applying twice a day to the skin affected, Athlete's foot was cured.

(Baldness)

The said spraying agent was sprayed on a bald part of a head. In this case, taking the typical CPL powder orally as well as applying ointment further enhances the effect of hair growth. Put it more concretely, hair growth is achieved by using the spray twice a day (10 g in terms of CPL powder) and taking 10 g/day orally for approximately 50 days.

<Effects of CPL on Humans>

(Actions of CPL on Human Bodies)

From results of studies, CPL is known to be a safe substance to the extent that its lethal dose cannot be determined; it causes no adverse reactions, and exists also in normal cells. From the above, it is known not to affect the results of examinations carried out after healthy subjects have ingested CPL (6 g/day) including physical findings such as auscultation, percussion, palpation, inspection, etc., general hematological/biochemical tests, test values for urine and faeces. Moreover, there are no abnormal findings at all from examinations carried out on patients who have taken 6 g/day for as long as 3-5 years. It has no effect on biogenic basic functions such as appetite, sleep, changes in weight, libido, etc. Rather, there are findings that seem to suggest the facilitation of such functions.

Findings of ameliorated adverse reactions, macrobiotic effects, and those that seem to suggest a cure were observed in the majority of several hundreds of patients suffering from cancer who have taken CPL (10-20 g/day). Although there is also an implication of the timing and period of administration, it is considered to be due to the fact that the liver/renal functions of these patients suffering from cancer are improved considering one of the actions of CPL, biogenic activator action, which is to enhance the functions of the liver, kidney and digestive system, activate the immune system, and improve these functions. Furthermore, it is considered to be applicable to patients suffering from intractable diseases such as liver/renal disorder, immune disease, etc. and in fact, similar satisfactory results have been obtained by administering it to these patients.

NK cells attached to cancer cells initially thrust perforin onto the wall of the cancer cells. Cell sap starts to leak from the cancer cells holed by this and the cells eventually become unable to operate and are killed. At this time, a liquid with a damaging action is released from granules inside NK cells and from this liquid, toxic oxygen called active oxygen is generated.

On the other hand, active oxygen is generated from oxygen in a reaction circuit called the electron transfer system attached to the citric acid circuit present in mitochondrion. This active oxygen moves inside cells, passes by the cornea, and reaches the gene DNA stored in the nucleus.

If the structure of the gene DNA deprived of the electron by the active oxygen is damaged, the structure of proteins generated based on the DNA may become abnormal. Abnormal proteins cause abnormal cells and furthermore, the functions of the organs become problematic. As a result, the cells most seriously become cancerous.

In addition, active oxygen generated in mitochondrion may attach to the cell membrane. The cell membrane also has the function of receiving extracellular information (role of a receptor) and transferring this information intracellularly.

If the cell membrane with these roles is damaged by active oxygen, the cells can no longer respond in a normal way to various stimulations and the signal transduction system is also damaged significantly. This damage results in a disease. As well as that we now know that active oxygen is involved in the facilitation of physiological aging, the harm of the active oxygen is critical for the state of pathology and diseases. Superoxide, hydrogen peroxide, and hydroxyl radical, which belong to the group of active oxygen, appear when energy is generated in intracellular mitochondrion. Diseases caused by damaged gene DNA and cell membranes include cancer, myocardial infarction, cerebral infarction, diabetes, allergic diseases, etc., which are lifestyle-related diseases with growing societal concerns. As this suggests, the active oxygen has a characteristic of a “double-edged sword”. Therefore, how this active oxygen is effectively utilized is critical for maintaining health and overcoming disease. Incidentally, cyclic polylactic acids are known to delete this active oxygen. Therefore, administering it to healthy people activates the cells and as a result, tissues, which improve functions.

Furthermore, although further examinations are necessary for more details, some studies have shown that it has a significant effect on correcting immune abnormalities, protecting the liver, adjusting blood sugar, maintaining muscular endurance, etc.

(Effects of CPL on Cancer Cells)

As one of the action mechanisms of CPL, it inhibits the activity of pyruvic acid kinase and lactate dehydrogenases (LDH), which are enzymes in the anaerobic glycolytic pathway playing a role in supplying energy to cancer cells, in particular, its action on inhibiting the LDH activity of cancer cells is significant. Morphologically, it causes vacuolation, and the expansion of the cytoplasm of cells, disintegration and aggregation of the nucleus. Furthermore, as a result of degenerating and weakening the cell as a whole including the cell membrane, it controls the proliferation of cancer cells and leads them to disappear. These are considered to inhibit not only the synthesis of ATP but also the actions of mRNA and be involved in the synthesis of RNA and DNA, and the generation of intracellular energy by acting on the metabolism of the intracellular organelle such as rough-surfaced endoplasmic reticulum, smooth-surfaced endoplasmic reticulum, ribosome, Golgi apparatus, etc., and as a result, controlling the metabolism of the cells, influencing the synthesis of proteins/glycoprotein, intracellular lipid metabolism, and movement and transport of ions. In other words, the metabolism of cancer cells is inhibited significantly. Morphologically, in the case that cells end up degenerating, the degeneration generally changes first to the nucleus followed by the cytoplasm. However, in the case that CPL is administered, pyknosis, disintegration, dissolution, etc. of cancer cells occur almost at the same time as the regressive degeneration of cytoplasm. This suggests that CPL inhibits the actions of the intracellular organelle significantly and influences the proliferation mechanism for the cells as a whole.

In addition to the action on the anaerobic glycolytic pathway, CPL is known to influence the activity of at least NK (Natural Killer) cells. The NK cells are one of the major immune cells to attach to cancer cells.

Furthermore, there is a clear correlation between the period of administration and the concentration of blood iron ions. That is, it has been confirmed that, the greater the dosage, and the longer the period of administration, the lower the concentration of blood iron ions.

This suggests that CPL incorporates blood iron ions into its cyclic structure and deprives the cancer cells of iron, which the cancer cells require as they are undergoing extremely rapid cell divisions. Thus, it inactivates them.

Other action mechanisms have also been indicated and CPL seems to exert its anticancer actions as a synthetic action of these.

(Effects of CPL On Other Tissues)

The fact that CPL inhibits the process of anaerobic metabolism of cancer cells seems to suggest that it also has, in the background, an action of some sort on the process of aerobic metabolism. As one of such actions, we considered effects on intracellular lipid metabolism and examined the effects of CPL ingestion on the metabolic process of blood serum and triglyceride, etc. in different organs and tissues.

CPL ingestion has an effect on the lipid metabolism system; in particular, it changes the fatty acid composition of triglyceride and causes desaturation as well as causes a change to the phospholipids composition, which constitutes the cells. This seems to suggest that it has some kind of influence on muscle training and CPL may operate to maintain muscular endurance.

The skeletal muscle, which operates our bodies, can be divided into the red muscle and white muscle. The white muscle is generally distributed near the surface of the body and operates sharply but can be easily exhausted. The red muscle is situated deeper in the body near the skeleton, which is suitable for long and endurable contractions. The metabolism of the red cell is aerobic and the activity of oxidase is high while the white cell has a high activity of the glycolytic enzymes.

Ca++ present in the sarcoplasmic reticulum is involved in the muscle contraction. Ca++ is released by means of a change in the membrane potential, which is in charge of adjusting the contraction reaction under the presence of Mg-ATP. As a direct energy source for the muscle contraction, ADP/AMP in addition to ATP is known to be necessary. The source of chemical changes in the muscle contraction is the decomposition of proteins, lipids and sugar and the energy source for the muscle exists particularly in lipids and sugar.

In the process of glycolysis, the process of metabolism from glycogen / dextrose to pyruvic acids is carried out oxygen-free. Inorganic phosphorus facilitates the glycolysis of glycogen during which ATP is generated. For muscle contraction, APT is used as an energy source, which turns into lactic acid with no supply of oxygen. When oxygen is supplied, it is re-synthesized into glycogen. Lipids are decomposed into glycerin and fatty acids and enter into the TCA cycle via acetyl Co-A to generate ATP.

As one of the action mechanisms of CPL, it activates the operations of the intracellular organelle, acts on the glycolytic pathway, TCA cycle, and respiratory system of the tissues, and is involved closely in the production of ATP, etc. Therefore, CPL is considered necessary for maintaining muscle endurance. It has also been revealed that CPL is necessary for the contraction of not only the skeletal muscles but the involuntary muscles called smooth muscles, which are distributed in the digestive tube, etc. in addition to the contraction/cell metabolism of myocardium.

Among those patients affected by intractable diseases, the bowel movement of those who were suffering from diarrhea, etc. improved or even constipation ameliorated for some during the ingestion of CPL. Subsequently, other patients suffering from constipation ingested CPL, which resulted in improved defecation and discontinuation of the use of a laxative. This suggests that CPL may improve also the movement and absorption of the intestinal tract, which is involved in constipation.

Biochemically and cytologically, the improvement of intracellular metabolism including the functions of the intracellular organelle, in particular, lipid metabolism, was observed. Absorbed substances were incorporated into the liver via the portal vein and decomposed, synthesized and accumulated in the liver. It can be considered that CPL improves these functions and further, the systemic functions.

From the above, CPL acts on various metabolic processes and in the case of abnormal cells/tissues of healthy people, it facilitates the functions. In the case of patients suffering from intractable diseases including immune diseases, liver/renal dysfunction, endocrine diseases, and malign tumors, it ameliorate/improves the symptoms by selectively acting on inhibiting abnormal metabolic processes and activating the functions of normal cells/tissues.

(A Role of CPL as a Functional Food—Cell Activator—)

CPL is excreted from cells in response to biogenic statuses and is one type of biogenic protective reaction however; its excretion is considered to be limited. Therefore, those with completely the same bioactivity were synthesized and incorporated it into the body, which improved cell functions or enhanced the functions in the case of normal cells, facilitated the activation of cells, inhibited the metabolic process in the case of abnormal cells and forced them to degenerate. Such a substance is necessary for maintaining health, preventing diseases, overcoming diseases and recovering. Furthermore, some studies suggest the possibility of the application to preventative medicine and sports medicine.

(CPL in terms of measurement values by the Magnetic lusupired Resonance Spectrum) All substances consist of atoms such as C, H, O, etc. and each atom emits different magnetic waves. Biogenic organs and their constituent cells, and substances such as food also emit specific magnetic waves as an aggregation of the waves of atoms. MIRS can catch these slight waves, estimate the health status of biogenic functions and the degree of diseases from the extent of derangement in these waves, and estimate whether or not the waves emitted by substances such as food are compatible with the organism. Because it can discern the status of organisms and substances at the atomic level as shown above, the application to the development of food, evaluation of effectiveness and adverse reactions, selection of toxic environmental chemical compounds to organisms, and even clinical diagnoses has began.

Therefore, CPL (cyclic polymerization lactic acid) as a health food was examined. In addition, changes in MIRS values in a patient suffering from lung cancer before and after taking CPL.

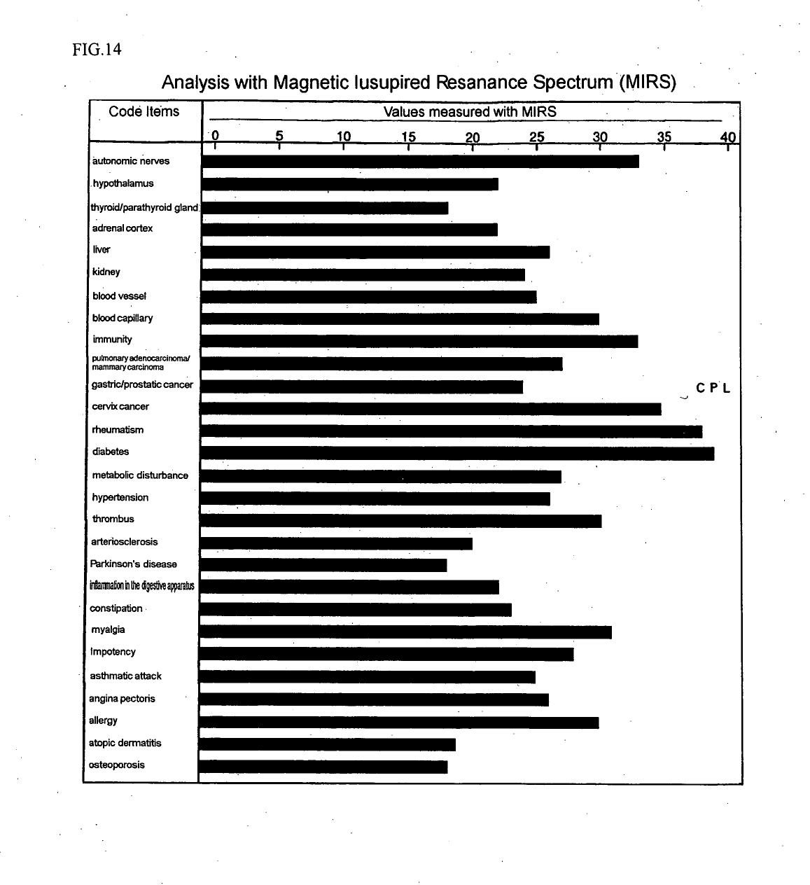

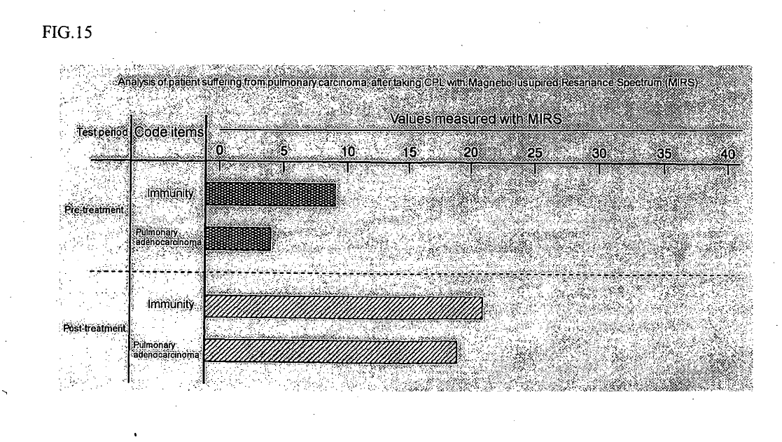

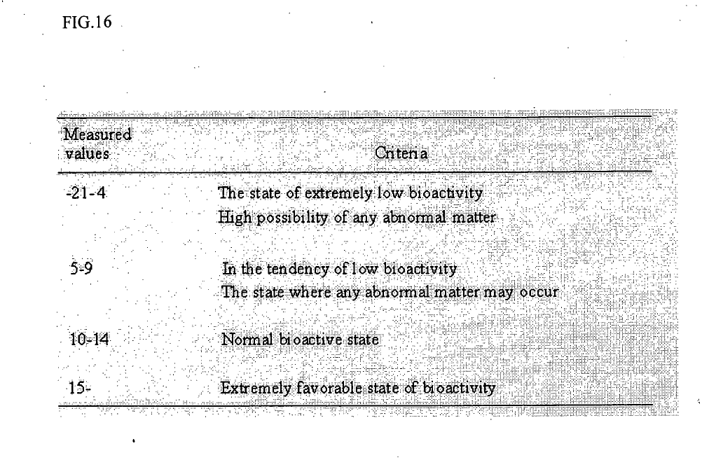

Values of CPL measured on MIRS are shown in FIG. 14, measurement values of MIRS in a patient suffering from lung cancer are shown in FIG. 15, and criteria for bioactivity by means of measurement values of MIRS are shown in FIG. 16.

For CPL, the MIRS values were high in general, which implied that the waves of the substance and organism resonate well (i.e., bio-compatibility and usefulness are high). In fact, taking CPL increased significantly the MIRS values in the patient suffering from lung cancer and improvement of the degree of bioactivity was confirmed.

<Examples of Administration To Bovines>

(Mastitis )

There are three types of CPL administration methods in bovine with mastitis: 1) oral-administration alone; 2) PL tester after injection of 50 cc of CPL ointment laced with 5 cc of dexamesone; 3) and combined administration via the oral route and in the form of ointment.

Complete response (CR) was achieved by compelling bovines to take 50 g of CPL (powder) dissolved in water 2 to 5 days after administration. When no response was observed after oral administration of CPL, PL tester (−) was applied after injection of CPL ointment laced with 5 cc of dexamesone. CR was achieved by orally administrating 50 g of CPL (powder) and applying CPL ointment for four days. If an antibiotic drug is administered as usual, it takes at least one week after CR to make shipment of milk from the bovines, resulting in loss of 70,000-100,000 yen per bovine. However, when administrating antibiotics with CPL, milk from the bovines could be shipped in about 2 to 5 days, and the loss was on a downward trend. In the bovines that underwent treatment using CPL alone, we could achieve a good result in that milking from the other normal udders were available even during treatment. Thus, the invention can contribute to dairy managerial economies.

(Antepartum, Protection of Dawner)

From 3 to 5 days before expected date of delivery, 50 g/day of CPL administration was initiated. In the group of bovines in which 50 g/day of CPL administration was initiated 5 days before the expected date of delivery, the bovines tended to give birth to calves smoothly and it seemed to promote the growth of the calves. Furthermore, it could be confirmed to play a role in mastitis prevention.

(Ketosis (Obese Syndrome))

For 10 days before the expected date of delivery, 100 g/day of mesothiol was administered in bovines with ketosis. Then, 100g/day of CPL in combination with 50 g/day was administered for 7 days after the delivery. The levels of GOT and γGTP, which were 549 and 100 in onset, respectively, remarkably decreased to 83 and 35, respectively when the symptom was improved.

(Pneumonia)

In the bovine with pneumonia, 20 g/day of CPL with was administered for 1-2 days and then the symptom improved (or recovered).

(Diarrhea in Calf)

In calves which developed diarrhea when changing to milk substitute, 20-30 g/day of CPL was administered and the calves recovered completely approximately 3 days after the CPL administration. Diarrhea frequently occurred in calves when changing to the milk substitute, which is thought to inhibit normal development of the calves. Then farmers may suffer a serious loss.

(Bacterial Diarrhea)

In the milking bovines, a bacterial diarrhea started to spread. Initially, four bovines infected by the bacterial diarrhea one after another. CPL 50 g/day was administered in all bovines that were infected or developed the bacterial diarrhea. In addition, 100 g/day of Neothor in combination with the CPL was administered in the bovines with severe diarrhea. As a result, complete response was achieved in most bovines infected with the bacterial diarrhea. The bovines fed as usual even in the condition of diarrhea and no decrease in the amount of milk was observed.

(Prevention of Disease)

Only CPL 10-20 g/day is necessary.

It is suggested that diarrhea can be prevented by administrating 20 g/day of CPL in calves for the first 4-5 days. For examples, diarrhea was spontaneously cured in the calves that took CPL and the calves grew up.

### INDUSTRIAL AVAILABILITY

(Utilization of CPL)

In the group of bovines with high CPK values, administration of CPL changed to extremely low mean values.

As shown in the CPK levels (muscle enzyme) of table 1 and 2, in the group of bovines with markedly high levels of CPK, CPL administration produced a change in CPK to extremely low mean level.

Blood screening test suggested some relief of abnormal inflammation. On the other hand, there were no specific changes in other blood responses such as WBC, GOT, and TCHO.

That is, CPL administration affects not normal cells but abnormal cells. This characteristics suggested that the effect of CPL on activities of normal cells without any side-effect can be expected.

TABLE 1

| | | Blood | | | | | | | | |
|---|---|---|---|---|---|---|---|---|---|---|
| 923 | | screening | Biochemical examination of blood | | | PL test | | | | |
| (Garget) | | WBC(×10²) | GOT(U/I) | TCHO(mg/d) | CPK(U/I) | □ | □ | □ | □ | Findings |
| Normal values (literary | | 40˜120 | 32˜105 | 60˜290 | 20˜320 | | | | | |
| reference) | | | | | | | | | | |
| Pretreatment | 20 April | 101 | 123 | 247 | 563 | + | + | + | | PL was implemented on 17 |
| values | | | | | | | | | | April |
| Initiation date | 30 April | | | | | | | | | (Spectrazol |
| of treatment | | | | | | | | | | From 2 May to 4 May) |
| | 3 May | 111 | 107 | 255 | 203 | | | | | |
| | 7 May | 97 | 81 | 208 | 133 | + | + | + | + | PL was implemented on 8 |
| | | | | | | | | | | May |
| Completion | 10 May | 110 | 107 | 259 | 121 | | | | | |
| date of | | | | | | | | | | |
| treatment | | | | | | | | | | |

TABLE 2

| | Blood | | | | | | | | |
|---|---|---|---|---|---|---|---|---|---|
| 901 | screening | Biochemical examination of blood | | | PL test | | | | |
| (Screening) | WBC(×10²) | GOT(U/I) | TCHO(mg/d) | CPK(U/I) | \textcircled{1} | \textcircled{2} | \textcircled{3} | \textcircled{4} | Findings |
| Normal values (literary | 40˜120 | 32˜105 | 60˜290 | 20˜320 | | | | | |
| reference) | | | | | | | | | |

| | | | | | |
|---|---|---|---|---|---|
| Pretreatment | 20 April | 97 | 134 | 255 | 819 |
| values | | | | | |
| Initiation date | 30 April | | | | |
| of treatment | | | | | |
| | 3 May | 96 | 128 | 266 | 82 |
| | 7 May | 94 | 116 | 245 | 97 |
| Completion | 10 May | 71 | 115 | 286 | 113 |
| date of | | | | | |
| treatment | | | | | |

(Effect of CPL Application To Bovine)

The use of CPL for garget and other diseases provided favorable unprecedented results in bovine. Our sequential investigation using bovines clarified that as well as the use of CPL alone, combination therapy of CPL and other agents will generate a synergistic effects and inhibit the side-effects of antibiotics.

CPL improves any disease by killing abnormal cells and malignant cells, and boosting the immune system. That is, even in the bovines with high sensitivity, relatively good results and possibilities can be expected among same domestic mammals. Although the amount of CPL is thought to be dozens of times of that of human due to the body weight of bovines, actual amount of CPL will be about the same as that of human because the effect of CPL as a cell activator depends on the molecular levels of cells and substances.

Introduction of CPL for early detection and treatment and prevention of bovine diseases will reduce the time required for treatment compared with the conventional treatments and promote phylactic system. In addition, possibility for reducing the loss for these diseases. That is, the us of CPL may have a favorable impact on the economy of dairy management.

1. A receiving apparatus, comprising:
demodulation means for demodulating a reception signal to a signal on a real axis and a signal on an imaginary axis;
carrier-to-noise (C/N) ratio calculation means for calculating a C/N ratio with amplitudes in an amplitude direction of signal points of the reception signal demodulated by said demodulation means and a C/N ratio with the amplitudes in a phase direction of the signal points of the reception signal demodulated by said demodulation means;
phase noise detection means for detecting phase noise based on the C/N ratio calculated with the amplitudes in the amplitude direction and the C/N ratio calculated with the amplitudes in the phase direction; and
indication means for indicating the C/N ratios calculated by said C/N ratio calculation means and the phase noise detected by said phase noise detection means.

2. The receiving apparatus as set forth in claim 1,
wherein said demodulation means comprises phase compensation means for compensating a phase with an external compensation signal, and
wherein when the phase noise takes place, said phase compensation means compensates the phase.

3. The receiving apparatus as set forth in claim 1,
wherein when the phase noise detected based on the C/N ratio calculated with the amplitudes in the amplitude direction and the C/N ratio calculated with the amplitudes in the phase direction is equal to or larger than a predetermine value, said indication means indicates an alarm.

4. The receiving apparatus as set forth in claim 1,
wherein said indication means indicates the phase noise detected based on the C/N ratio calculated with the amplitudes in the amplitude direction and the C/N ratio calculated with the amplitudes in the phase direction as a numeric value.

5. A carrier-to-noise (C/N) ratio indication method for a receiving apparatus, the method comprising the steps of:
demodulating a reception signal to a signal on a real axis and a signal on an imaginary axis;
calculating a C/N ratio with amplitudes in an amplitude direction of signal points of the reception signal demodulated by said demodulation means and a C/N ratio with amplitudes in a phase direction of the signal points of the reception signal demodulated by said demodulation means;
determining whether phase noise takes place based on the C/N ratio calculated with the amplitudes in the amplitude direction and the C/N ratio calculated with the amplitudes in the phase direction; and
indicating the C/N ratios calculated by said C/N ratio calculation means and the phase noise determined in said step of determining.

6. The C/N ratio indication method for a receiving apparatus as set forth in claim 5,
wherein when the phase noise determined based on the C/N ratio calculated with the amplitudes in the amplitude direction and the C/N ratio calculated with the amplitudes in the phase direction is equal to or larger than a predetermine value, said indication means indicates an alarm.

7. The C/N ratio indication method for a receiving apparatus as set forth in claim 5,
wherein said indication means indicates the phase noise determined based on the C/N ratio calculated with the amplitudes in the amplitude direction and the C/N ratio calculated with the amplitudes in the phase direction as a numeric value.
